(19) United States
(12) Patent Application Publication
(10) Pub. No.: US 2016/0331588 A1
(43) Pub. Date: Nov. 17, 2016

(54) SCHLEMM'S CANAL STENT-SIEVE

(71) Applicant: UNIVERSITY OF UTAH RESEARCH FOUNDATION, Salt Lake City, UT (US)

(72) Inventors: Balamurali Ambati, Salt Lake City, UT (US); Alan Crandall, Salt Lake City, UT (US); Bruce K. Gale, Salt Lake City, UT (US); Christopher Lambert, Salt Lake City, UT (US)

(21) Appl. No.: 15/111,757

(22) PCT Filed: Jan. 14, 2015
(86) PCT No.: PCT/US15/11394

§ 371 Date: Jul. 14, 2016

Related U.S. Application Data
(60) Provisional application No. 61/927,051, filed on Jan. 14, 2014.

(51) Int. Cl. A61F 9/007; A61F 2/82

(52) U.S. Cl. CPC A61F 9/00781; A61F 2/82; A61F 2230/0006; A61F 2210/0076

(57) ABSTRACT

An ocular stent device (100) can include an elongate arcuate body portion (102) configured in size and shape to be inserted into the Schlemm's canal. The body portion (102) can be inserted along an arc of at least 100 degrees of the Schlemm's canal. The body portion (102) can include a plurality of through-holes (104) adapted to direct flow of aqueous humor from a trabecular meshwork to episcleral collector channels of the eye.

### RELATED APPLICATION

This application claims the benefit of U.S. Provisional Application No. 61/927,051, filed January 14, 2014, which is incorporated herein by reference.

### BACKGROUND

Glaucoma is the second leading cause of blindness in the United States and in the world, affecting almost 3 million Americans and 70 million patients globally. The most common cause of glaucoma is elevated eye pressure. When eye drop medications fail to control elevated eye pressure, surgery is often utilized. Trabeculectomy is often prescribed to reduce intraocular pressure. Trabeculectomy is a surgical procedure that removes part of the trabecular meshwork of the eye and adjacent structures. This allows drainage of aqueous humor from within the eye to underneath the conjunctiva where the aqueous humor is absorbed. Although improved techniques and the adjunctive use of antimetabolites have enhanced long-term success as measured by intraocular pressure control, trabeculectomy still has a sizeable risk profile.

Due to complications associated with trabeculectomy, a variety of devices, including aqueous shunts, are being evaluated as alternative surgical treatments for patients with inadequately controlled glaucoma. Micro-stents are also being evaluated in patients with mild to moderate open-angle glaucoma currently treated with ocular hypotensive medication. Some examples of glaucoma surgical implant devices include the Molteno implant, the Baerveldt® implant, the Ahmed tube shunt, and more recently the Express-Shunt, mini-Express shunt, canaloplasty, iStent®, Cypass Micro-Stent®, and Hydrus. Although the primary indication for most implants is the failure of primary medical or surgical therapy, some ophthalmologists have advocated use of implants as a primary intervention. Most devices do not preclude subsequent trabeculectomy if needed. Older versions of such surgical devices involved large surgeries with high risk of infection. Newer micro surgeries have been developed with lower risk, but these have proven to be considerably less effective at lowering intraocular pressure.

### SUMMARY

An ocular stent device can include an elongate arcuate body portion configured in size and shape to be inserted into the Schlemm's canal. The body portion can be inserted along an arc of at least 100 degrees of the Schlemm's canal. Also, the body portion can include a plurality of through-holes oriented to direct flow of aqueous humor from a trabecular meshwork to episcleral collector channels of the eye.

In some embodiments of the stent device, the body portion can have an interior lumen extending along at least a partial length of the body portion. In further optional embodiments, the body portion can have an insertion end with a tip biased toward an inner circumferential surface of the body portion such that the tip forms an acute angle with the inner circumferential surface of the body portion. Typically, the body portion can be formed from a compliant material such as a polymeric material or flexible alloy.

In yet another embodiment, the through-holes can extend along hole axes which extend from an inner circumferential surface of the body portion to an outer circumferential surface. In some examples, the hole axes can be coplanar with a device plane coincident with the body portion. In other examples, the hole axes can be non-coplanar with the device plane.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A is a schematic showing a top transparent view a stent device in accordance with one embodiment of the present invention.

FIG. 1B is an exploded view of a body section of FIG. 1A.

FIG. 1C is a side cross-sectional view taken along line 1C of FIG. 1A.

FIG. 1D is an exploded cross-sectional view taken from FIG. 1C showing a hole axis non-planar with a device plane.

FIG. 1E is an exploded view of tips of FIG. 1A.

FIG. 2 is a schematic illustrating a rounded tip of a stent device in accordance with an embodiment of the present invention.

FIG. 3 is a schematic showing elliptical through-hole openings in accordance with an embodiment of the present invention.

FIG. 4 is a flow chart showing a method of relieving intraocular pressure using a stent device in accordance with an embodiment of the present invention.

FIG. 5A is a front view of an eye having an incision therein to allow insertion of a stent device in accordance with one embodiment of the present invention.

FIG. 5B is a front view of the eye of FIG. 5A showing the stent device partially inserted into the Schlemm's canal in accordance with one embodiment of the present invention.

FIG. 5C is a front view of the eye of FIG. 5B showing the stent device fully inserted into the Schlemm's canal in accordance with one embodiment of the present invention.

FIG. 6A is a side cross-sectional view of an eye.

FIG. 6B is an exploded view taken from FIG. 6A illustrating tissue regions adjacent the Schlemm's canal.

FIG. 6C is a further exploded view of FIG. 6B illustrating a stent device and associated fluid flow across the Schlemm's canal in accordance with one embodiment of the present invention.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

As used herein, anatomical direction terms such as “anterior,” “posterior,” “superior,” and “inferior” describe directions or locations with respect to a subject into which a stent device may be implanted. These terms are used with their normal meanings in the ocular field and anatomical arts.

As used herein, “through-hole” refers to a hole or channel that extends from one exterior surface of a stent device through the stent device to another exterior surface, such that the openings of the hole at each surface are coaxial with each other. Specifically, the openings are coaxial along a hole axis which is typically substantially perpendicular to a longitudinal axis of the elongated body. As such, the hole openings at least partially cross such a hole axis, and are in most cases each centered along the hole axis. In cases where the stent device is solid (non-luminal), a through-hole extends straight from one surface to another opposite surface. In cases where the stent device is luminal, a through-hole refers to a first opening at an external surface of the device, the hole or channel extending through the stent wall to the lumen, and then a second hole or channel in the opposite stent wall extending to a second opening aligned along the hole axis at an opposite external surface. Thus, a through-hole in a luminal stent device includes two openings in the stent wall that are coaxial with each other. Although the through-hole extends “straight through” the device (meaning that the openings at either end are coaxial), the profile of the hole is not necessarily linear from one end to the other. For example, a through-hole can be flared such that one opening has a larger diameter than the other opening. In other cases, the diameter of the through-hole can vary in other ways along the length of the through-hole. For example, hole profiles can include straight (constant cross-section), flared inward, flared outward, hour-glass shape, and the like.

As used herein, “major diameter” refers to the diameter of the circle or arc formed by the body portion of the stent device, which approximately matches the diameter of the Schlemm's canal and the iris of the eye as measured to a center of the canal or center of the body portion. “Major radius” refers to one half of the major diameter.

As used herein, “minor diameter” refers to the diameter or thickness of the tube used to form the body portion. In some cases the body portion can be formed of a tube (with or without a lumen) with a circular cross-section, and the minor diameter is the diameter of the circular cross-section. In other cases the body portion can be an elongate body with an elliptical, square, or other-shaped cross-section. In these cases, the minor diameter is the longest dimension across the cross-section. “Minor radius” refers to one half of the minor diameter.

It is noted that, as used in this specification and in the appended claims, the singular forms “a,” “an,” and “the” include plural referents unless the context clearly dictates otherwise. Thus, for example, reference to “a through-hole” includes one or more of such features, reference to “an end” includes reference to one or more of such elements, and reference to “inserting” includes reference to one or more of such steps.

As used herein, the terms “about” and “approximately” are used to provide flexibility, such as to indicate, for example, that a given value in a numerical range endpoint may be “a little above” or “a little below” the endpoint. The degree of flexibility for a particular variable can be readily determined by one skilled in the art based on the context.

As used herein, the term “substantially” refers to the complete or nearly complete extent or degree of an action, characteristic, property, state, structure, item, or result. The exact allowable degree of deviation from absolute completeness may in some cases depend on the specific context. However, the nearness of completion will generally be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Schlemm's Canal Stent-Sieves

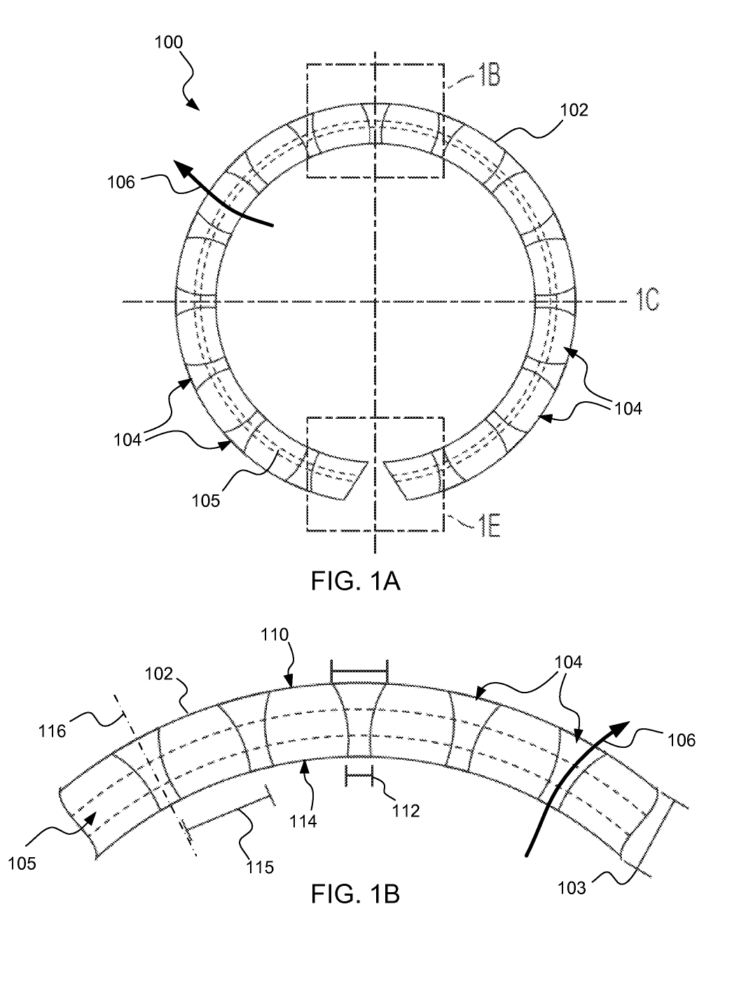

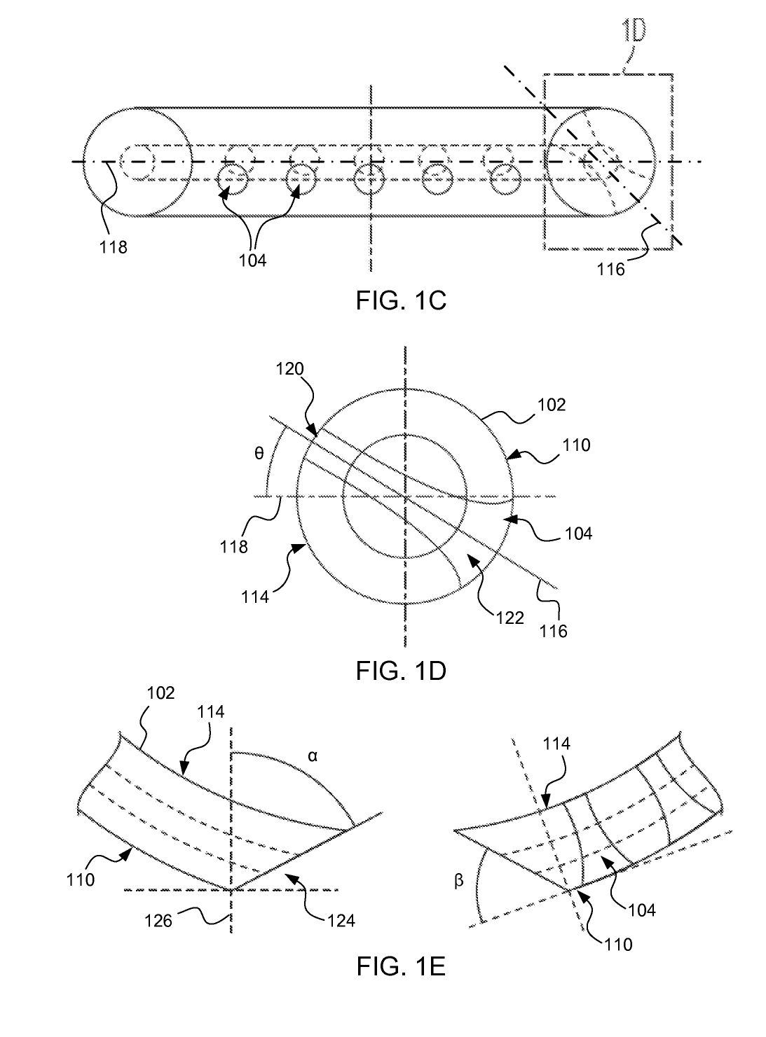

Referring generally to FIG. 1A, a stent device 100 is illustrated which is insertable into a Schlemm's canal in an eye of a patient. The stent device can include an elongate arcuate body portion 102 which is configured in size and shape to be inserted into the Schlemm's canal as described in more detail herein. The arcuate body portion can extend along an arc of at least 100 degrees. The arc of the body portion is intended to correspond to a common arc within the Schlemm's canal upon insertion of the device. Also, the body portion can include a plurality of through-holes 104 which direct flow 106 of aqueous humor from a trabecular meshwork to episcleral collector channels of the eye. Generally, the body portion can be an elongated tube with or without an interior lumen.

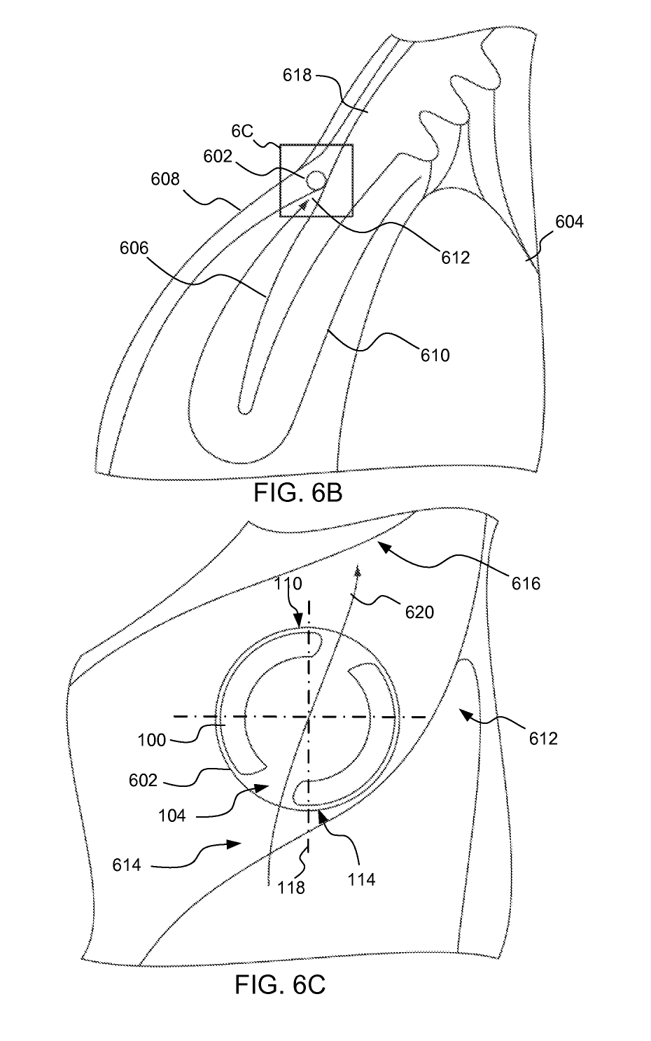

FIG. 6A illustrates a simplified view of a typical eye 600 in cross-section. Specifically, the Schlemm's canal 602 is located anterior to the lens capsule 604 and near the junction of the iris 606 with the cornea 608. FIG. 6B shows this region in greater detail along with a fluid flow 610 of aqueous humor from posterior regions, towards anterior regions of the eye and the Schlemm's canal and specifically the angle 612 of the anterior chamber of the eye. The trabecular meshwork 614 includes tissue generally located between the angle 612 and the Schlemm's canal 602, while episcleral collector channels 616 are located generally opposite the Schlemm's canal. FIG. 6C illustrates a stent device 100 oriented within the Schlemm's canal 602.

Referring now to FIG. 1B, the body portion 102 can have a minor diameter 103 (i.e. an exterior diameter of the cross-section of the elongate body portion) which allows for insertion into the Schlemm's canal. The minor diameter can be small enough to be inserted into the Schlemm's canal without causing substantial tissue damage, but large enough to hold the Schlemm's canal open sufficient to increase fluid flow through the Schlemm's canal. Typically, the body portion can have a minor diameter from about 100 microns to about 500 microns, and in some cases from 200 microns to about 400 microns, although other dimensions can be useful. In one specific example a minor diameter of 300 microns or 360 microns can be used.

The body portion can be curved to approximately match the curvature of a Schlemm's canal. Thus, the major diameter of the body portion can be about the same as the diameter of the Schlemm's canal, which is also about the same as the diameter of the iris. The elongate body portion can also have a major radius (i.e. the radius of the arc of at least 180 degrees of the Schlemm's canal) which generally aligns with a Schlemm's canal of a patient. This radius may vary slightly among patients, although a major radius from about 4.5 mm to about 10 mm is widely useful, and in most cases about 5 mm to about 7 mm can be used, making the major diameter of the body portion about 9 mm to about 20 mm. Body portions with diameters within this range can approximately match the major diameter of the Schlemm's canal of a patient. It should be noted that the major diameter of the Schlemm's canal can vary between patients, including those with microcornea, megalocornea, or animals with glaucoma. To accommodate for varying sizes of Schlemm's canals, the stent device can be made with an average major diameter and the body portion can be flexible so that the major diameter of the body portion can expand or contract slightly to match the Schlemm's canal of the patient. In one particular embodiment, the major diameter can be about 12 mm. Alternatively, the Schlemm's canal of the patient can be measured and then a stent device can be custom made to match the particular patient.

The body portion can have various lengths. The arcuate body portion can extend along an arc which is sufficient to affect flow of aqueous humor toward the episcleral collector channels. Generally, the body portion can be long enough to extend along an arc of at least 100 degrees of the Schlemm's canal, and in some cases at least 180 degrees. In one example, the body portion can extend along an arc of at least 270 degrees corresponding to a similar portion of the Schlemm's canal. In another example, the body portion can be configured to be inserted into the Schlemm's canal along an arc from about 330 degrees to about 360 degrees. A specific embodiment is shown in FIG. 1 in which the body portion extends along about 360 degrees of the Schlemm's canal, encircling the entire iris.

The body portion can have any suitable length such as from about 8 mm to about 70 mm, although 10 mm to about 40 mm can be useful. In one specific example, the body portion can be from about 36 mm to about 38 mm long. In another particular embodiment, the body portion can be about 30 mm long. In some cases multiple shortened stent devices can be inserted at various locations along the Schlemm's canal as part of a segmented stent system. Such segmentation can allow a greater degree of flexibility for insertion and customization for specific patients. For example, segmentation can be particularly useful for patients exhibiting a septated Schlemm's canal, e.g. congenital glaucoma, uveitic glaucoma, Axenfeld-Rieger syndrome, or prior glaucoma surgery. Segmented stent system can typically include two to four segments spaced 1-2 mm apart and utilizing the same overall dimensions previously discussed.

In some embodiments of the stent device, the body portion can have an interior lumen 105 extending along at least a partial length of the body portion. The lumen can improve flow of aqueous humor through the stent device, for example by allowing fluid to flow freely among the plurality of through-holes 104. In one aspect, the interior lumen can extend an entire length of the body portion such that each end is also open to allow fluid communication from the lumen to exterior regions through each end. The lumen can have a lumen diameter that is less than the minor diameter of the body portion. The interior lumen can generally have a diameter which is from 20% to 75% of the minor diameter, and in some cases from 40% to 60%. For example, in one embodiment the lumen can have a diameter from about 100 microns to about 200 microns. In one particular example, the lumen can have a lumen diameter of 150 microns. However, in alternative embodiments the body portion can be non-luminal, having void space only along through-holes. In some cases, the body portion can be a substantially homogeneous solid material throughout, except for the through-holes. Alternatively, the body portion can be formed of a porous or mesh material.

Typically, the body portion can be formed from a biocompatible polymeric material. Non-limiting examples of suitable polymeric material can include silicone, polytetrafluoroethylene, perfluoroalkoxy, fluorinated ethylene-propylene, carbothane, polyurethanes, polyethylenes, silicone elastomer, polyimide, polypropylene, acrylic and collamer (collagen/poly-HEMA), and combinations thereof. In one example, silicone, polypropylene, and polytetrafluoroethylene can be particularly useful as the polymeric material. In another alternative aspect, the body portion can be formed of a bioerodible material. Non-limiting examples of suitable bioerodible materials can include poly(lactic-co-glycolic acid), polycaprolactone, polyglycolic acid, polylactic acid, polyethylene glycol polymers, and the like. However, biocompatible metal alloys can also be suitable. In one optional aspect, the body portion can be formed from a compliant metal. Non-limiting examples of compliant metals can include Nitinol (nickel-titanium alloy such as Nitinol 55, Nitinol 60, etc.), and the like.

The material can be flexible to allow the body portion to flex while being inserted into the Schlemm's canal and also to allow the major diameter of the body portion to expand or contract to match the diameter of a patient's Schlemm's canal. A flexible material can also have shape memory to allow the body portion to maintain a ring shape. Further, the material can be sufficiently stable in situ to allow for permanent or long-term use with minimal deterioration. In one particular example, the body portion can be formed by placing tubing, such as PFA tubing, in a circular shaping mold while heating the tubing to 100° C. This creates a flexible body portion that can hold a ring shape through shape memory.

Biocompatible coatings can be applied to the body portion to improve long-term biocompatibility and/or fluid flow. For example, the biocompatible coating can include agents capable of minimizing fibroblast migration and proliferation as well as avoiding inflammation. Fluid flow can be improved by using a coating that increases wettability of the body portion. Non-limiting examples of materials useful in the biocompatible coating can include albumin, rapamycin, polyethylene glycol, tacrolimus, anti-fibrotic drugs, anti-fouling agent (e.g. VITROSTEALTH commercially available from DSM), fibronectin, and combinations thereof.

While the stent device acts to open a collapsed or otherwise occluded Schlemm's canal, the through-holes act as channels for fluid to flow freely across the Schlemm's canal. Each through-hole extends straight through the body portion, providing a pathway for aqueous humor to flow from the trabecular meshwork to the episcleral collector channels. In embodiments with an interior lumen, the lumen can also allow aqueous humor to flow between the plurality of through-holes. The through-holes can extend along hole axes which extend from an inner circumferential surface of the body portion to an outer circumferential surface. The through-holes can be arranged in a variety of ways to improve flow of aqueous humor. As shown in the figures, the through-holes can extend out radially from the center of the arc of the body portion.

The through-holes can be formed by any suitable method, such as drilling, machining, laser cutting, or other methods. In one particular example, the through-holes can be cut into the body portion using a laser. In some embodiments, the through-holes can have a substantially uniform diameter along the length of the through-holes. However, in some cases the through-holes can have a cross-section which varies along a hole length. For example, the through-holes can be flared so that one opening of the through holes has a greater diameter than the other opening. Referring to FIG. 1B, although not required, the through-holes 104 can be flared such that the through-holes have an outer diameter 108 which is greater at an outer circumferential surface 110 of the body portion 102 than an inner diameter 112 at an inner circumferential surface 114 of the body portion. Outward flaring of the through-holes can enhance flow and reduce occurrence of occlusions.

The through-holes can also have a variety of sizes. Typically, the difference from inner diameter to outer diameter of flared holes can be from 140% to 200%, although other differences may be suitable. The through-holes can also have a variety of sizes. In some embodiments, the through-holes can have a diameter from about 50 microns to about 200 microns. In one particular embodiment, the through holes can have a diameter of 100 microns. As explained above, the through-holes can also have different diameters at each opening. FIG. 1B shows an example of through-holes with a diameter of 100 microns on the inner circumferential surface of the body portion, and a diameter of 150 microns on the outer circumferential surface.

Furthermore, the through-holes 104 can be spaced around the circumference of the body portion. For example, the through-holes can be evenly spaced or non-uniformly spaced. The plurality of through-holes can also be spaced around the entire circumference of the body portion, or only around a section of the body portion. In one particular embodiment, the plurality of through-holes can be evenly spaced around substantially the entire circumference of the body portion. Spacing of the through-holes can also affect fluid flow towards the episcleral collector channels. In some cases it can be desirable to customize the rate fluid flow by adjusting through-hole spacing, depending on severity of excess IOP. Spacing the through-holes around the entire circumference of the device can enable a surgeon to insert the device anywhere around the limbus without having to be concerned about alignment of fluid flow with the location of the collector channels that drain the fluid. The spacing between the through holes can also vary. Generally, through-holes can be spaced apart by a distance 115 of about 400 microns to about 600 microns. As a general guideline, the hole distance between adjacent through-holes can be maintained from 0.5 to 10 times a largest hole diameter, and in some cases from 1 to 6 times. In one particular embodiment, the plurality of through-holes can be evenly spaced around substantially an entire length of the body portion.

In alternative embodiments, the hole axes can be oriented at an angle with respect to the device plane. Although other configurations can be used, the hole axes can each be angled at a common angle with respect to the device plane. Thus, the hole axes can be oriented at varied angles along the stent device. The through-holes 104 can extend along hole axes 116 which extend from the inner circumferential surface 114 of the body portion 102 to the outer circumferential surface 110. In some examples, the hole axes can be coplanar with a device plane 118 coincident with the body portion. In other examples, the hole axes can be non-coplanar with the device plane. FIG. 1C illustrates through-holes 104 which have a hole axis 116 which is non-parallel to the device plane 118. As shown in FIG. 1D, the hole axis 116 can be offset from the device plane 118 by an angle θ. The offset angle can orient an inner opening 120 closer to trabecular tissue, while an outer opening 122 can be oriented closer to episcleral tissue as compared to a hole axis which is parallel to the device plane. Specifically, FIG. 1D is oriented with the left side being an inner circumferential surface 114 of the ocular stent, the right side being an outer circumferential surface 110 of the ocular stent, and the upper region facing an anterior region of an eye upon insertion. Referring back to FIG. 6C, the stent device 100 is shown rotated approximately 90 degrees counter-clockwise. Accordingly, as the stent device is inserted into the Schlemm's canal 602 the through-holes 104 angle backward as the through-holes extend radially outward from the center of the device arc. The through-holes on the inner side of the ring can be slightly superior (i.e. facing the anterior chamber), while hole entrances on the outer side of the ring can be slightly inferior (i.e. facing collector channels 616 draining the Schlemm's canal). This oblique angle of entry facilitates flow from the anterior chamber through the trabecular meshwork 614 to the collector channels 616. In other words, the openings of the through-holes at the outer circumferential surface 110 of the body portion are posterior with respect to the openings at the inner circumferential surface 114. Such an arrangement can more closely match the openings of the through holes to the locations of the trabecular meshwork 614 and the episcleral collector channels 616. In other embodiments, the through-holes can be flared in the opposite direction so that the diameter is greater at the inner circumferential surface.

Referring again to FIG. 1D, the hole axes 116 can be oriented at an angle θ from about 0 degrees to about 180 degrees with respect to the device plane 118. In some such embodiments, the hole axes can be oriented at an angle from about 10 degrees to about 60 degrees with respect to the device plane. In one particular example, the hole axes can be oriented at an angle of about 30 degrees with respect to the device plane. Thus, in some embodiments, the hole axes can be non-planar with the device plane.

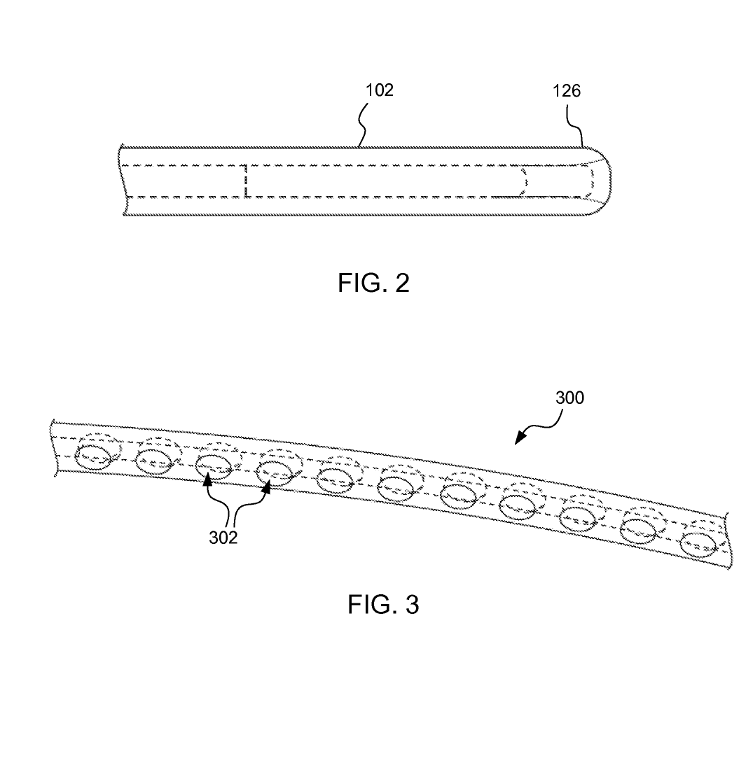

Referring now to FIG. 1E, the body portion 102 can have at least one insertion end 124 with a tip biased toward the inner circumferential surface 114 of the body portion such that the tip surface forms an acute angle β with the outer circumferential surface 110 of the body portion. Similarly, a complimentary angle a is formed between an axes 126 which is perpendicular to the outer circumferential surface and the tip. For example, in some embodiments the angle θ can be from about 30 degrees to about 60 degrees. In one specific example, the angle θ can be about 45 degrees. In one alternative, both ends can include the biased tip. The biased tip can facilitate penetration through the Schlemm's canal during insertion and avoid damaging an outer circumferential wall of the Schlemm's canal. In some cases an opposing end can have a flat end (i.e. angle of 0 degrees) or a rounded tip. Specifically, FIG. 2 illustrates rounded tips 126 which can be formed on one or both ends of the elongate body portion 102.

Although the through-holes can often have generally circular cross-section, this is not required. FIG. 3 illustrates a portion of a stent device 300 with through-holes 302 having an elliptical cross-section. The same principles apply to this through-hole configuration as discussed herein for alternatively shaped through-holes.

The stent device described herein can be used to treat glaucoma in patients having elevated intraocular pressure. The Schlemm's canal and the trabecular meshwork account for about ninety percent of the aqueous outflow in the human eye. As previously noted with respect to FIG. 6A through 6C, these structures are located at the junction between the iris 606 and the sclera 618. The region within the anterior chamber at this junction or corner is known as “the angle” or iridial angle 612. The trabecular meshwork 614 is a wedge-shaped structure that runs around the circumference of the eye. The outer wall of the trabecular meshwork coincides with the inner wall of Schlemm's canal 602 which is a tube-like structure that runs around the circumference of the cornea 608.

In patients with open-angle glaucoma, the cause of elevated intraocular pressure is believed to be a collapsing of the Schlemm's canal 602 and blockage of the trabecular meshwork 614. The stent-sieve device 100 as described herein can open the Schlemm's canal 602 and allow flow 620 of fluid from the trabecular meshwork 614 across the Schlemm's canal 602 and into the episcleral collector channels 616 around the Schlemm's canal. The stent device can provide multiple flow channels for fluid drainage from the eye, thus relieving intraocular pressure.

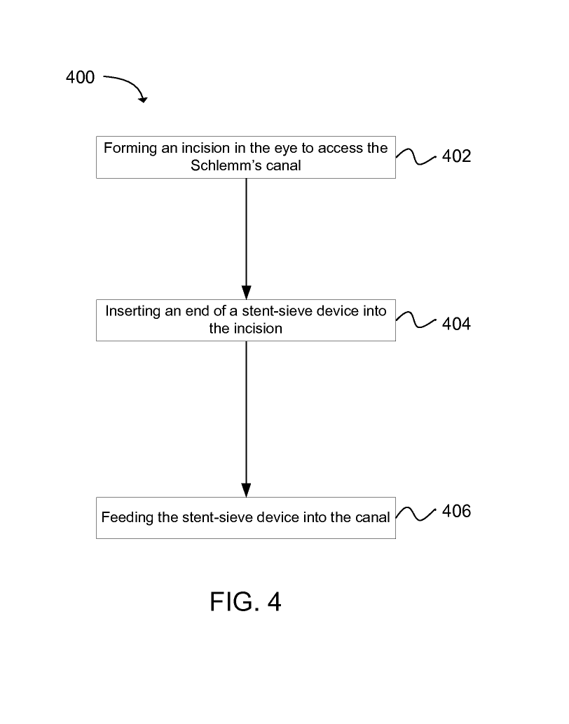

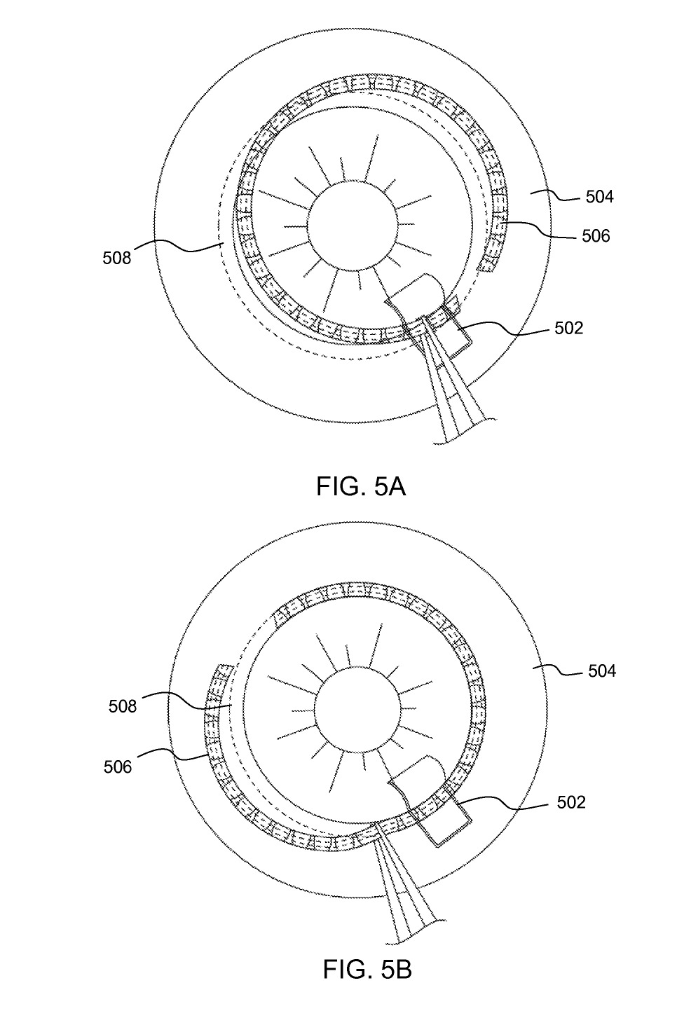

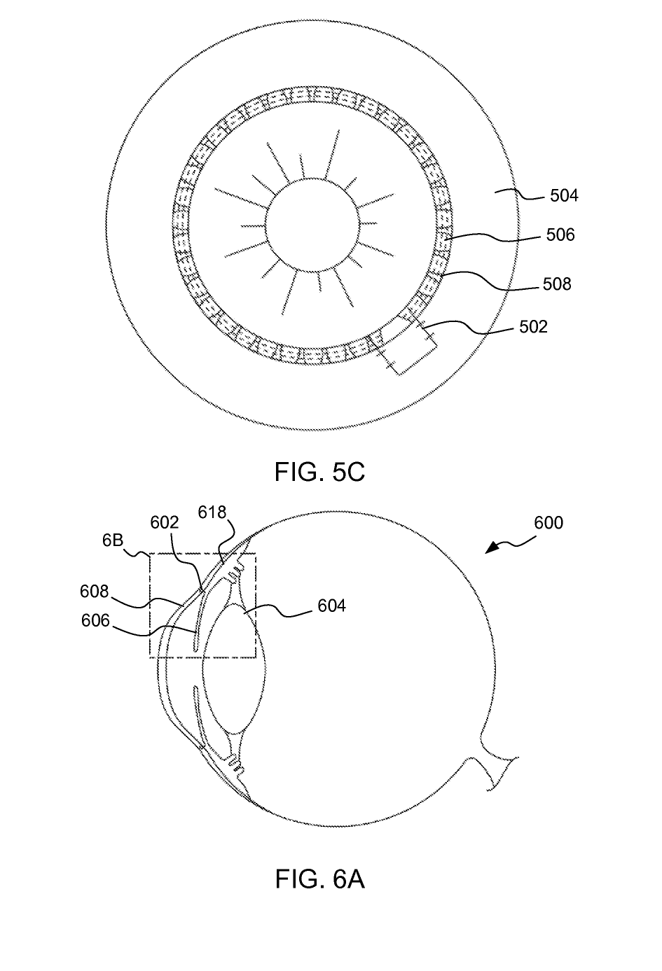

Thus, as outlined by FIG. 4 and generally illustrated by FIGS. 5A through 5C, a method 400 of reducing intraocular pressure can include implanting the stent device in a Schlemm's canal of a patient by a surgeon. The stent device can be inserted into the Schlemm's canal using any suitable surgical approach. Non-limiting examples include manual surgical manipulation or an injector, either externally (e.g. through scleral-cut-down) or internally (e.g. through anterior chamber under visualization of the angle with a gonioscopic lens). Specifically, the surgeon can form an incision in the eye 402 to provide access to the Schlemm's canal. FIG. 5A illustrates the incision 502 via sclera-cut-down in the eye 504. The incision can be in the cornea, corneoscleral limbus (i.e. border between the cornea and the sclera) through a scleral cut-down under a scleral flap ab externo (referred to as an external approach) or into the angle under gonioscopic visualization ab interno (referred to as an internal approach), or other suitable access tissue. The method can also include inserting 404 one end of the stent device 506 into the Schlemm's canal 508 through the incision 502. As illustrated in FIG. 5B, the surgeon can then feed 406 (see FIG. 4) the remaining length of the device 506 into the Schlemm's canal 508 until the entire stent device is within the Schlemm's canal as illustrated in FIG. 5C. Accordingly, at least one of the ends of the body portion of the stent device can be configured as an insertion end. This insertion end can have a tip shaped in a variety of ways to improve insertion. An angled tip can improve ease of insertion of the stent device by providing a narrow insertion point at the inner circumferential surface side of the tip while reducing the likelihood of catching on the Schlemm's canal wall at the outer circumferential surface side of the tip. Once the stent is fully inserted, the incision can be closed and optionally sutured.

In some embodiments, both ends of the body portion can be insertion ends with angled tips, as shown in FIGS. 1A and 1E. This can allow a surgeon to insert the stent device in either direction based on specific patient and surgical conditions. In other embodiments, one of the ends can be a non-insertion end. The non-insertion end can have a flat tip (forming 90 degree angles with the surfaces of the body portion) or a rounded tip as previously described in connection with FIG. 2.

### EXAMPLE 1

A Schlemm's canal stent-sieve was fabricated by placing a 30 mm length of PFA tubing (manufactured by Upchurch Scientific and purchased through IDEX Health and Science) in a circular shaping mold and heating the tubing to 100° C. to form a body portion with a 12 mm diameter. The body portion had a minor diameter of 360 microns and a lumen diameter of 150 microns. Through-holes were formed using a CO₂ laser system (Universal Laser Systems VLS3.60). The through-holes were made with a diameter of 100 microns and spaced about 400 microns apart. The hole axes were coplanar with the device plane of the body portion.

### EXAMPLE 2

The Schlemm's canal stent-sieve was tested by insertion into two cadaver eyes to confirm insertability of the device and flowability of fluids across the device. Tryptan blue was injected into the anterior chamber and coloring was observed to enter the conjunctiva confirming flow across the stent device.

### EXAMPLE 3

The stent-sieve of Example 1 was inserted into an eye of a live rabbit. The conjunctiva was cut near the upper fornix exposing the sclera. A deep sclerotomy was performed exposing an area of the Schlemm's canal. The stent implant was intended to be threaded through an entire length of the Schlemm's canal but encountered significant resistance at approximately ¼ revolution around the canal upon insertion. The implant was removed and two pieces were cut at approximately ¼ the circumference yielding two quarter ring implants. One quarter ring implant was inserted in each direction of the exposed Schlemm's canal opening. The scleral flap was subsequently sutured and both rabbit eyes (one operated and one un-operated) received topical dexamethasone for one week.

Intraocular pressure (IOP) was measured daily in both eyes for two weeks and no significant pressure differences were detected between the operated and un-operated eyes. The average pressure in the implanted eye was 12.0±1.3 mm Hg while the average pressure in the eye with no implant was 11.9±1.2 mm Hg. Thus, the stent did not raise IOP in a normal rabbit eye, which was expected from the normal baseline pressure.

The rabbit was then euthanized and the eyes were enucleated, sectioned, and stained with hemotoxylin and eosin. The analysis revealed minimal fibrous tissue and no inflammation around the site of the implant which evidenced good biocompatibility.

The described features, structures, or characteristics may be combined in any suitable manner in one or more examples. In the preceding description numerous specific details were provided, such as examples of various configurations to provide a thorough understanding of examples of the described technology. One skilled in the relevant art will recognize, however, that the technology may be practiced without one or more of the specific details, or with other methods, components, devices, etc. In other instances, well-known structures or operations are not shown or described in detail to avoid obscuring aspects of the technology.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

1. An ocular stent device comprising an elongate arcuate body portion configured in size and shape to be inserted into a Schlemm's canal of an eye along an arc of at least 100 degrees of the Schlemm' s canal, the body portion including a plurality of through-holes configured to direct flow of aqueous humor from a trabecular meshwork to episcleral collector channels of the eye.

2. The stent device of claim 1, wherein the body portion has a minor diameter from about 200 microns to about 400 microns.

3. The stent device of claim 1, wherein the body portion has a major radius from about 5 mm to about 7 mm.

4. The stent device of claim 1, wherein the body portion is from about 36 mm to about 38 mm long.

5. The stent device of claim 1, wherein the body portion has an interior lumen extending along at least a partial length of the body portion.

6. The stent device of claim 5, wherein the lumen has a diameter from about 100 microns to about 200 microns.

7. The stent device of claim 1, wherein the body portion is non-luminal.

8. The stent device of claim 1, wherein the body portion is configured to be inserted into the Schlemm's canal along an arc of at least 270 degrees of the Schlemm's canal.

9. The stent device of claim 1, wherein the body portion is configured to be inserted into the Schlemm's canal along an arc from about 330 degrees to about 360 degrees of the Schlemm's canal.

10. The stent device of claim 1, wherein the body portion has an insertion end having a tip biased toward an inner circumferential surface of the body portion such that the tip forms an acute angle with the inner circumferential surface of the body portion.

11. The stent device of claim 10, wherein the angle is from about 30 degrees to about 60 degrees.

12. The stent device of claim 1, wherein the body portion is formed from a polymeric material.

13. The stent device of claim 1, wherein the body portion is formed from a compliant metal.

14. The stent device of claim 1, wherein the body portion is coated with a biocompatible coating.

15. The stent device of claim 1, wherein the through-holes extend along hole axes which extend from an inner circumferential surface of the body portion to an outer circumferential surface.

16. The stent device of claim 15, wherein the hole axes are coplanar with a device plane coincident with the body portion.

17. The stent device of claim 15, wherein the hole axes are oriented at an angle from about 0 degrees to about 180 degrees with respect to a device plane coincident with the body portion.

18. The stent device of claim 17, wherein the angle is from about 10 degrees to about 60 degrees.

19. The stent device of claim 1, wherein the through-holes are flared such that the through-holes have a greater diameter at an outer circumferential surface of the body portion than at an inner circumferential surface of the body portion.

20. The stent device of claim 1, wherein the through-holes have a diameter from about 50 microns to about 200 microns.

21. The stent device of claim 1, wherein the through-holes are spaced from about 400 microns to about 600 microns apart.

22. The stent device of claim 1, wherein the plurality of through-holes are evenly spaced around substantially an entire circumference of the body portion.

23. A method of reducing intraocular pressure in an eye, comprising:
a) forming an incision in the eye to access the Schlemm's canal; and
b) inserting the stent device of claim 1 into the Schlemm's canal.

24. The stent device of claim 1, wherein the body portion is an elongated tube.
